(19) United States
(12) Patent Application Publication
(10) Pub. No.: US 2011/0054504 A1
(43) Pub. Date: Mar. 3, 2011

(54) RECANALIZATION DEVICE WITH EXPANDABLE CAGE

(75) Inventor: Stephen C. Porter, Oakland, CA (US)

(73) Assignee: BOSTON SCIENTIFIC SCIMED, INC., Maple Grove, MN (US)

(21) Appl. No.: 12/870,485

(22) Filed: Aug. 27, 2010

Related U.S. Application Data
(60) Provisional application No. 61/238,344, filed on Aug. 31, 2009.

(51) Int. Cl. A61B 17/22

(52) U.S. Cl. 606/159

(57) ABSTRACT

A vascular recanalization device for re-establishing blood flow through a vessel lumen. The vascular recanalization device includes an elongate wire and an expandable cage slidably coupled to the elongate wire. The expandable cage includes a proximal collar slidably disposed on the elongate wire and a distal collar slidably disposed on the elongate wire. A central stop is secured to the elongate wire intermediate the proximal collar and the distal collar. A proximal stop is located proximal of the proximal collar of the expandable cage, and a distal stop is located distal of the distal collar of the expandable cage. The proximal collar is slidable along the elongate wire between the proximal stop and the central stop, and the distal collar is slidable along the elongate wire between the central stop and the distal stop.

### RELATED APPLICATION DATA

The present application claims the benefit under 35 U.S.C. §119 to U.S. provisional patent application Ser. No. 61/238,344, filed Aug. 31, 2009. The foregoing application is hereby incorporated by reference into the present application in its entirety.

### TECHNICAL FIELD

The disclosure is directed to a medical device for treatment of a body vessel. More particularly, the disclosure is directed to a vascular recanalization device having an expandable cage for re-establishing blood flow through a vessel lumen.

### BACKGROUND

Acute ischemic stroke is a fast onset disease with the potential for devastating long-term neurological effects, or even death. Treatment of patients which might yield a significant beneficial effect should be done rapidly in order to re-establish blood flow to the affected region of the brain before unrecoverable damage has occurred. One such method is the placement of a stent-like device across an embolic blockage in order to rapidly re-establish blood flow. However, permanent placement of a stent may be undesirable in at least some instances.

There is an ongoing need, therefore, to provide alternative configurations of vascular recanalization devices for temporary placement in a vessel lumen to rapidly re-establish blood flow through the vessel lumen.

### SUMMARY

The disclosure is directed to several alternative designs, materials and methods of manufacturing and using medical device structures and assemblies.

Accordingly, one illustrative embodiment is a medical device including an elongate wire and an expandable cage coupled to the elongate wire such that the expandable cage is longitudinally slidable along a length of the elongate wire between a first position and a second position. The medical device further includes a central stop secured to the elongate wire intermediate the proximal end and the distal end of the expandable cage. The proximal end of the expandable cage is prevented from sliding distally beyond the central stop, and the distal end of the expandable cage is prevented from sliding proximally beyond the central stop.

Another illustrative embodiment is a vascular recanalization device for re-establishing blood flow through a vessel lumen. The vascular recanalization device includes an elongate wire and an expandable cage slidably coupled to the elongate wire. The expandable cage includes a proximal collar slidably disposed on the elongate wire and a distal collar slidably disposed on the elongate wire. A central stop is secured to the elongate wire intermediate the proximal collar and the distal collar. A proximal stop is located proximal of the proximal collar of the expandable cage, and a distal stop is located distal of the distal collar of the expandable cage. The proximal collar is slidable along the elongate wire between the proximal stop and the central stop, and the distal collar is slidable along the elongate wire between the central stop and the distal stop.

Yet another illustrative embodiment is a method of treating a vessel lumen. The method includes providing a medical device including an elongate wire, an expandable cage slidably coupled to the elongate wire, the expandable cage including a proximal collar slidably disposed on the elongate wire and a distal collar slidably disposed on the elongate wire, and a central stop secured to the elongate wire intermediate the proximal collar and the distal collar. The elongate wire is pushed distally, whereby the central stop abuts the distal collar of the expandable cage and is spaced away from the proximal collar, and the elongate wire is pulled proximally, whereby the central stop abuts the proximal collar of the expandable cage and is spaced away from the distal collar.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a plan view of an exemplary vascular recanalization device;

FIG. 1A is a cross-sectional view taken along line 1A-1A of FIG. 1;

FIGS. 2A-2C illustrate an exemplary mode of operating the vascular recanalization device of FIG. 1;

FIG. 3 is a plan view of another exemplary vascular recanalization device;

FIGS. 4A-4C illustrate an exemplary mode of operating the vascular recanalization device of FIG. 3;

FIG. 5 is a plan view of yet another exemplary vascular recanalization device;

FIGS. 6A-6F illustrate an exemplary mode of operating the vascular recanalization device of FIG. 5; and

FIGS. 7-10 illustrate various exemplary embodiments of an expandable mesh which may be used in the expandable cage of the vascular recanalization devices shown in FIGS. 1, 3 and 5.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used herein the terms “pull”, “pulling”, and “pulled” are intended to refer to applying a force to a leading end portion of an object in order to move the object in a direction generally toward the leading end portion of the object.

As used herein the terms “push”, “pushing”, and “pushed” are intended to refer to applying a force to a trailing end portion of an object in order to move the object in a direction generally away from the trailing end portion of the object.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

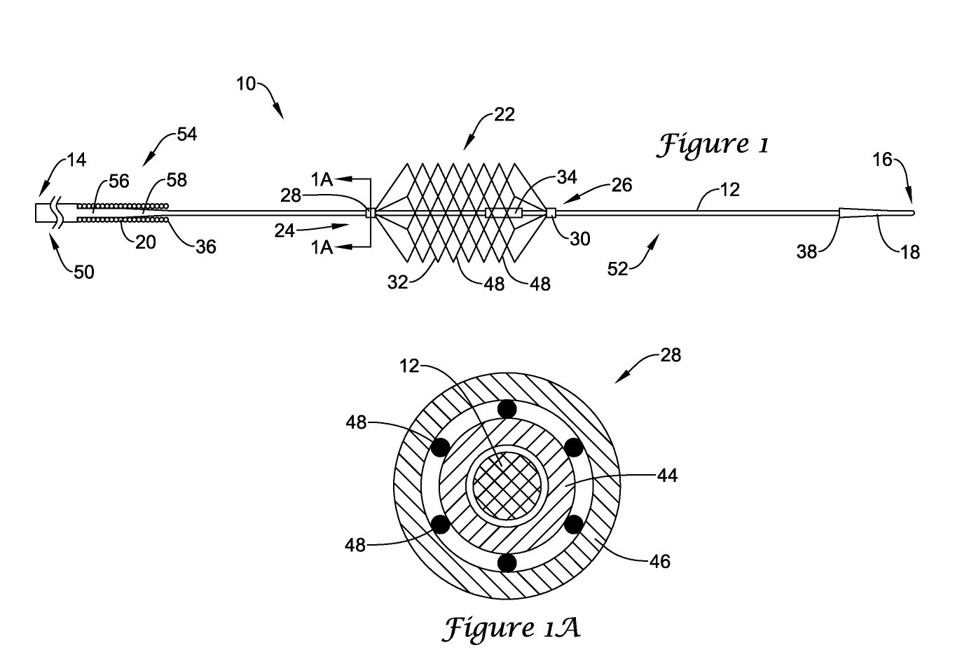

Referring to FIG. 1, there is shown a vascular recanalization device 10 which may be temporarily placed in a blocked vessel to re-establish blood flow through the vessel. The vascular recanalization device 10 may include an elongate wire 12 having a proximal end 14 and a distal end 16. In operation, the distal end 16 of the elongate wire 12 may be advanced through the vasculature of a patient while the proximal end 14 remains exterior of the patient to be manipulated by the medical personnel during a medical procedure.

The distal end 16 of the elongate wire 12 may include a distal tip, such as a distal coil tip 18 attached to the elongate wire 12. For example, the distal coil tip 18 may include a wire filament helically wound into a coil. The coil may be disposed over a distal portion of the elongate wire 12 and secured to the elongate wire 12, such as by welding, soldering, brazing, or adhesive bonding in some instances. In other embodiments, the distal tip of the elongate wire 12 may have a different configuration, if desired.

The elongate wire 12 may include a proximal region 50 (e.g., a proximal constant diameter region) having a first diameter, a distal region 52 (e.g., a distal constant diameter region) having a second diameter less than the first diameter, and a transition region 54 which varies in diameter from the first diameter of the proximal region 50 to the second diameter of the distal region 52. For instance, the transition region 54 may be tapered from the first diameter to the second diameter. The transition region 54 may include one or more tapers and/or step-wise transitions. As shown in FIG. 1, the transition region 54 may include a constant diameter portion 56 distal of the proximal region 50 and a tapered portion 58 extending between the constant diameter portion 56 and the distal region 52. The constant diameter portion 56 may have a third diameter less than the first diameter of the proximal region 50. The tapered portion 58 may have a variable diameter which varies from the third diameter of the constant diameter portion 56 of the transition region 54 to the second diameter of the distal region 52. However, in other embodiments, the transition region 54 may include a continuous taper from the first diameter of the proximal region 50 to the second diameter at the distal region 52, or two or more tapered sections separated by one or more constant diameter sections, for example.

The vascular recanalization device 10 may include a flexible tubular member 20, such as a coiled member or a slotted hypotube, disposed over the elongate wire 12 throughout at least a portion of the transition region 54. The flexible tubular member 20 may help provide a gradual transition in flexibility from the proximal region 50 to the distal region 52 of the elongate wire 12, and thus may help prevent kinking of the elongate wire 12. As shown in FIG. 1, a proximal portion of the flexible tubular member 20 may be disposed over and/or secured to the constant diameter portion 56 of the transition region 54 such that the proximal end of the flexible tubular member 20 is adjacent the distal end of the proximal region 50 of the elongate wire 12. The flexible tubular member 20 may also include a distal portion which extends over the tapered portion 58 of the transition region 54. It is noted that in embodiments in which the flexible tubular member 20 has a constant diameter, the distal portion of the flexible tubular member 20 may be spaced away from the tapered portion 58, providing an annular gap between the tapered portion 58 of the elongate wire 12 and the flexible tubular member 20. In some embodiments, the outer diameter of the flexible tubular member 20 may be substantially equal to the first diameter of the proximal region 50 of the elongate wire 12 to effect a smooth transition between the proximal region 50 and the flexible tubular member 20. In some embodiments, the distal end of the flexible tubular member 20 may be secured to the elongate wire 12.

The vascular recanalization device 10 may also include an expandable cage 22 coupled to the elongate wire 12. For instance, the expandable cage 22 may be slidably coupled to the distal region 52 of the elongate wire 12 to allow longitudinal translation of the elongate wire 12 relative to the expandable cage 22 while the expandable cage 22 remains stationary. In some embodiments the expandable cage 22 may be slidably coupled to the elongate wire 12 such that the proximal end 24 of the expandable cage 22 is slidably coupled to the elongate wire 12 and/or the distal end 26 of the expandable cage 22 is slidably coupled to the elongate wire 12. In some embodiments, no portion of the expandable cage 22 is fixedly secured to the elongate wire 12 or any other component of the vascular recanalization device 10.

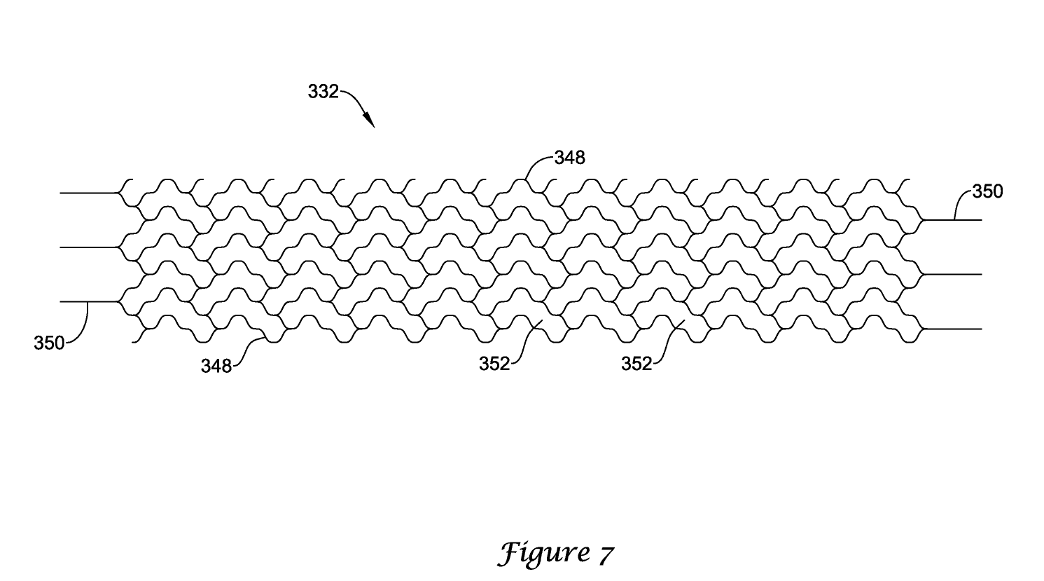

In some embodiments, as shown in FIG. 1, the expandable cage 22 may include a proximal collar 28 proximate the proximal end 24 of the expandable cage 22, a distal collar 30 proximate the distal end 26 of the expandable cage 22, and an expandable mesh 32 extending between the proximal collar 28 and the distal collar 30 and secured to both the proximal collar 28 and the distal collar 30. The expandable mesh 32 may be formed of any desired structure, including but not limited to those illustrative structures shown in FIGS. 7-10, herein. For instance the expandable mesh 32 may include a plurality of interconnected filaments 48 forming an annular framework having interstitial openings between adjacent filaments 48. In some instances the filaments 48 may be individual helically wound, braided or woven strands, or the filaments 48 may be portions of a workpiece remaining subsequent removal of material from the workpiece to form the interstitial openings. For example, the filaments 48 may be portions of a flat sheet or a tubular member remaining after removal of material from the flat sheet or tubular member.

The proximal collar 28 and/or the distal collar 30 may be slidably and rotatably disposed on the distal region 52 of the elongate wire 12. FIG. 1A, which is a cross-sectional view taken along line 1A-1A of FIG. 1, illustrates one possible configuration of the proximal collar 28 of the expandable cage 22. It is noted that the distal collar 30 may have a similar structure to that of the proximal collar 28, thus discussion of the structure of the proximal collar 28 may apply equally to that of the distal collar 30.

The collar 28, 30 may include an inner tubular portion 44 and an outer tubular portion 46 extending circumferentially around the elongate wire 12 and slidable thereon. For example, the inner tubular portion 44 may have an inner diameter slightly larger than the outer diameter of the distal region 52 of the elongate wire 12 to allow sliding and rotational movement of the collar 28, 30 over the elongate wire 12. One or more, or a plurality of filaments 48 of the expandable mesh 32 may extend into and/or through the collar 28, 30 between the inner tubular portion 44 and the outer tubular portion 46 such that the filaments 48 may be secured to the collar 28, 30. In other embodiments, the expandable mesh 32 may be secured to the proximal and distal collars 28, 30 in another manner, if desired.

The vascular recanalization device 10 may further include a central stop 34 secured to the elongate wire 12 at a location intermediate the proximal end 24 and the distal end 26 of the expandable cage 22. For example, the central stop 34 may be secured to the elongate wire 12 intermediate the proximal collar 28 and the distal collar 30 of the expandable cage 22. The expandable mesh 32 may circumferentially surround and be spaced away from the central stop 34. In some embodiments, the central stop 34 may be a helical coil member or other tubular member disposed around the elongate wire 12, or the central stop 34 may be one or more projections formed around or secured to the elongate wire 12.

The central stop 34 may be provided to prevent the expandable cage 22 from sliding distally on the elongate wire 12 distally of the central stop 34 and may be provided to prevent the expandable cage 22 from sliding proximally on the elongate wire 12 proximally of the central stop 34. For instance, the central stop 34 may prevent the proximal end 24 of the expandable cage 22 from sliding distal of the central stop 34 and the central stop 34 may prevent the distal end 26 of the expandable cage 22 from sliding proximal of the central stop 34. Thus, in the illustrative example of the vascular recanalization device 10 shown in FIG. 1, the central stop 34 may prevent the proximal collar 28 from sliding distal of the central stop 34, and the central stop 34 may prevent the distal collar 30 from sliding proximal of the central stop 34.

The vascular recanalization device 10 may also include a proximal stop 36 located proximally of the central stop 34 and a distal stop 38 located distally of the central stop 34. In some embodiments, the proximal stop 36 may be the distal end of the flexible tubular member 20, or the proximal stop 36 may be another component of the vascular recanalization device 10. In some embodiments, the distal stop 38 may be the proximal end of the distal coil tip 18, or the distal stop 38 may be another component of the vascular recanalization device 10.

The proximal collar 28, and thus the proximal end 24 of the expandable cage 22, may be longitudinally slidable along the elongate wire 12 between the proximal stop 36 and the central stop 34, and the distal collar 30, and thus the distal end 26 of the expandable cage 22, may be longitudinally slidable along the elongate wire 12 between the central stop 34 and the distal stop 38. The longitudinal distance of travel of the proximal collar 28 between the proximal stop 36 and the central stop 34 may be two, three, four, five, eight or ten times or more of the length of the proximal collar 28, in some instances. In some embodiments the proximal collar 28 may have 1 mm or more, 2 mm or more, 3 mm or more, 4 mm or more, 5 mm or more, 6 mm or more, 7 mm or more, 8 mm or more, 9 mm or more, or 10 mm or more of travel between the proximal stop 36 and the central stop 34. The longitudinal distance of travel of the distal collar 30 between the distal stop 38 and the central stop 34 may be two, three, four, five, eight or ten times or more of the length of the distal collar 30, in some instances. In some embodiments the distal collar 30 may have 1 mm or more, 2 mm or more, 3 mm or more, 4 mm or more, 5 mm or more, 6 mm or more, 7 mm or more, 8 mm or more, 9 mm or more, or 10 mm or more of travel between the distal stop 38 and the central stop 34.

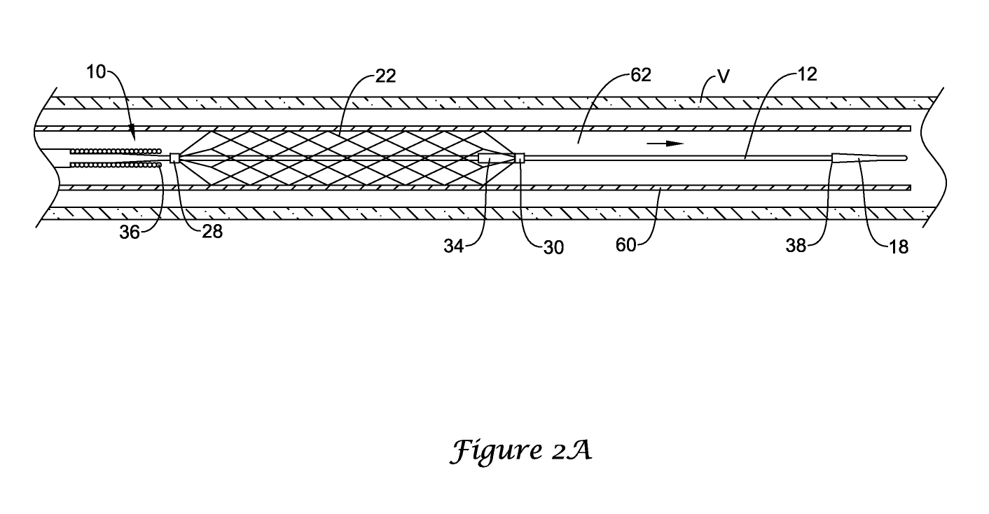

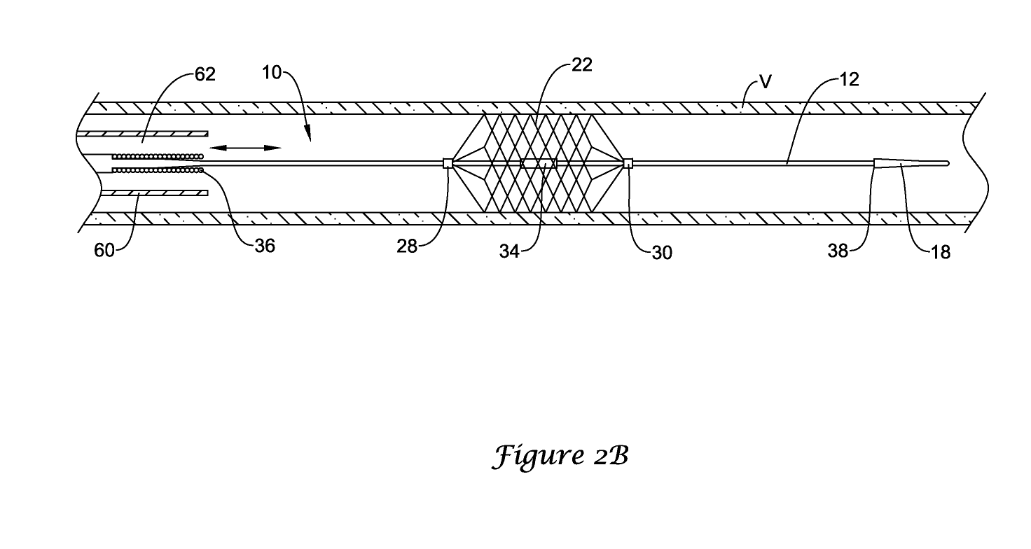

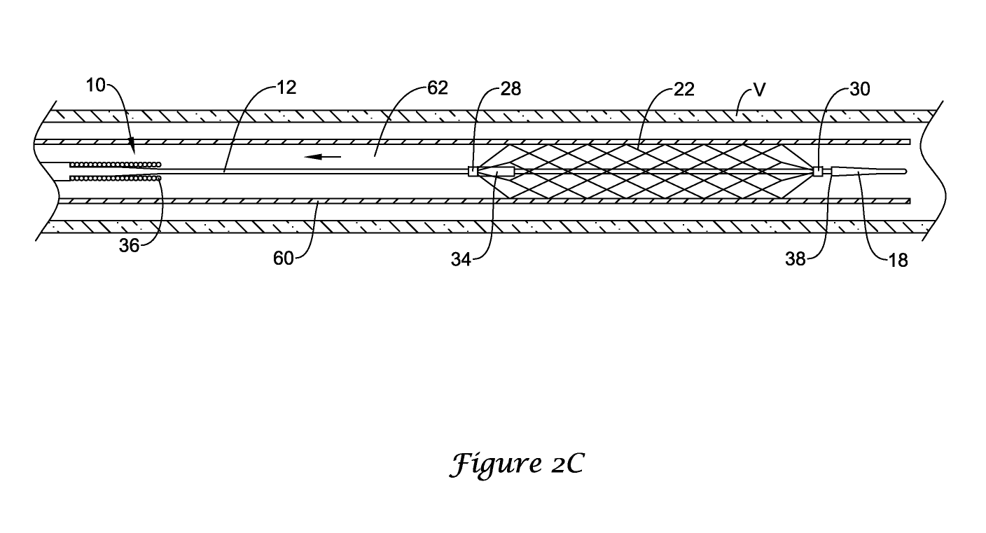

FIGS. 2A-2C illustrate an exemplary mode of operating the vascular recanalization device 10 of FIG. 1 during a medical procedure to re-establish blood flow through a vessel lumen. As shown in FIG. 2A, the vascular recanalization device 10 may be advanced distally through the lumen 62 of an elongate tubular member 60 of a medical device, such as a medical catheter (e.g., a microcatheter), during a medical procedure. For instance, the medical personnel may push the elongate wire 12 distally to advance the vascular recanalization device 10 through the elongate tubular member 60 located in a lumen of a vessel V.

While positioned in the lumen 62 of the elongate tubular member 60 the expandable cage 22 may assume a collapsed configuration in which the expandable cage 22 has a reduced diameter relative to an expanded configuration which the expandable cage 22 may be expanded to upon exiting the elongate tubular member 60. In some embodiments, the elongate tubular member 60 may constrain the expandable cage 22 to the collapsed configuration while positioned in the lumen 62 of the elongate tubular member 60. In the collapsed configuration, the expandable cage 22 may have a collapsed cage length measured from the proximal collar 28 to the distal collar 30. Furthermore, in the expanded configuration, shown in FIG. 2B, the expandable cage 22 may have an expanded cage length measured from the proximal collar 28 to the distal collar 30. The collapsed cage length may be greater than the expanded cage length.

As shown in FIG. 2A, the distance between the proximal stop 36 and the distal end of the central stop 34 may be equal to or greater than the collapsed cage length in order to allow the expandable cage 22 to be fully collapsed and elongated to the collapsed configuration while being advanced distally through the elongate tubular member 60. Furthermore, as shown in FIG. 2C, the distance between the proximal end of the central stop 34 and the distal stop 38 may be equal to or greater than the collapsed cage length in order to allow the expandable cage 22 to be fully collapsed and elongated to the collapsed configuration while being withdrawn proximally through the elongate tubular member 60.

As the elongate wire 12 is being pushed distally by the medical personnel, the central stop 34 moves distally relative to the expandable cage 22 until the distal end of the central stop 34 abuts the distal collar 30 of the expandable cage 22. Thus, the central stop 34 exerts a force on the distal collar 30, in which the exerted force has a directional component in a distal direction parallel to the longitudinal axis of the elongate wire 12, effectively resulting in the expandable cage 22 being pulled distally through the elongate tubular member 60 via the distal collar 30 by pushing the elongate wire 12 distally.

Effectively pulling the expandable cage 22 through the elongate tubular member 60 has the effect of reducing the force necessary to move the expandable cage 22 through the elongate tubular member 60 relative to situations in which the expandable cage 22 would be pushed through the elongate tubular member 60. Pulling the expandable cage 22 distally from the distal collar 30 will tend to cause the expandable cage 22 to lengthen and thus tend to decrease in diameter, reducing frictional forces between the expandable cage 22 and the inner surface of the elongate tubular member 60, whereas pushing the expandable cage 22 distally from the proximal collar 28 would tend to cause the expandable cage 22 to shorten and thus tend to enlarge in diameter, increasing frictional forces between the expandable cage 22 and the inner surface of the elongate tubular member 60.

The vascular recanalization device 10 may be advanced distally until the expandable cage 22 is deployed from the distal end of the elongate tubular member 60 to deliver the expandable cage 22 to a target location within the vessel V. As shown in FIG. 2B, once deployed from the elongate tubular member 60, the expandable cage 22 may be expanded to the expanded configuration in which the expandable cage 22 is expanded in diameter and shortened in length. In some embodiments, the expandable cage 22 may be self-expanding in which the expandable cage 22 automatically expands upon being deployed from the elongate tubular member 60. In other embodiments, the expandable cage 22 may be manually, selectively and/or controllably expanded, such as by the manipulation of a component of the vascular recanalization device 10, application of a stimulus, and/or activation/deactivation of an energy source, for example. In the expanded configuration, the expandable cage 22 may exert a radially outward force on the vessel V to open a blockage and re-establish blood flow through the vessel V. The openness of the interstitial openings between filaments 48 of the expandable mesh 32 allows blood to flow through the expandable cage 22. Furthermore, in some embodiments, the expandable mesh 32 may be configured to capture embolic material while deployed in the vessel V.

When the expandable cage 22 is in its expanded configuration and deployed in the vessel V, the elongate wire 12 may be longitudinally translated through the expandable cage 22 and/or rotated while the expandable cage 22 stays stationary and engaged against the vessel V, allowing the expandable cage 22 to float on the distal region 52 of the elongate wire 12. Thus, longitudinal movement of the elongate wire 12 up to a threshold amount, whether inadvertent or intentional, will not cause the expandable cage 22 to move.

For instance, in the expanded configuration, the elongate wire 12 is free to move longitudinally between a first position in which the central stop 34 abuts the distal collar 30 and is spaced away from the proximal collar 28 and a second position in which the central stop 34 abuts the proximal collar 28 and is spaced away from the distal collar 30. Thus, the elongate wire 12 is free to move longitudinally a distance equal to the distance between the proximal collar 28 and the distal collar 30 minus the length of the central stop 34. This distance may be considered the float length of the expandable cage 22 on the elongate wire 12. In some embodiments, the float length may be 1 mm or more, 2 mm or more, 3 mm or more, 4 mm or more, 5 mm or more, 6 mm or more, 7 mm or more, 8 mm or more, 9 mm or more, 10 mm or more, 15 mm or more, 20 mm or more, or 30 mm or more, for example.

The length of the central stop 34 may be any desired length. In some embodiments, the length of the central stop 34 may be 5% or less, 10% or less, 15% or less, 20% or less, 25% or less, 50% or less, 75% or less, 80% or less, 85% or less, 90% or less, or 95% or less of the distance between the proximal collar 28 and the distal collar 30 when the expandable cage 22 is in the expanded configuration. In some embodiments, the length of the central stop 34 may be 5% or more, 10% or more, 15% or more, 20% or more, 25% or more, 50% or more, 75% or more, 80% or more, 85% or more, 90% or more, or 95% or more of the distance between the proximal collar 28 and the distal collar 30 when the expandable cage 22 is in the expanded configuration. In some embodiments, the central stop 34 may have a length of about 1 mm, about 2 mm, about 3 mm, about 4 mm, or about 5 mm, for example.

As shown in FIG. 2C, the vascular recanalization device 10 may be withdrawn proximally through the lumen 62 of an elongate tubular member 60 of a medical device, such as a medical catheter (e.g., a microcatheter), at the completion of the medical procedure. For instance, the medical personnel may pull the elongate wire 12 proximally to withdraw the vascular recanalization device 10 through the elongate tubular member 60.

As the elongate wire 12 is being pulled proximally by the medical personnel, the central stop 34 moves proximally relative to the expandable cage 22 until the proximal end of the central stop 34 abuts the proximal collar 28 of the expandable cage 22. Thus, the central stop 34 exerts a force on the proximal collar 28, in which the exerted force has a directional component in a proximal direction parallel to the longitudinal axis of the elongate wire 12, effectively resulting in the expandable cage 22 being pulled proximally through the elongate tubular member 60 via the proximal collar 28 by pulling the elongate wire 12 proximally.

Effectively pulling the expandable cage 22 through the elongate tubular member 60 has the effect of reducing the force necessary to move the expandable cage 22 through the elongate tubular member 60 relative to situations in which the expandable cage 22 would be pushed through the elongate tubular member 60. Pulling the expandable cage 22 proximally from the proximal collar 28 will tend to cause the expandable cage 22 to lengthen and thus tend to decrease in diameter, reducing frictional forces between the expandable cage 22 and the inner surface of the elongate tubular member 60, whereas pushing the expandable cage 22 proximally from the distal collar 30 would tend to cause the expandable cage 22 to shorten and thus tend to enlarge in diameter, increasing frictional forces between the expandable cage 22 and the inner surface of the elongate tubular member 60.

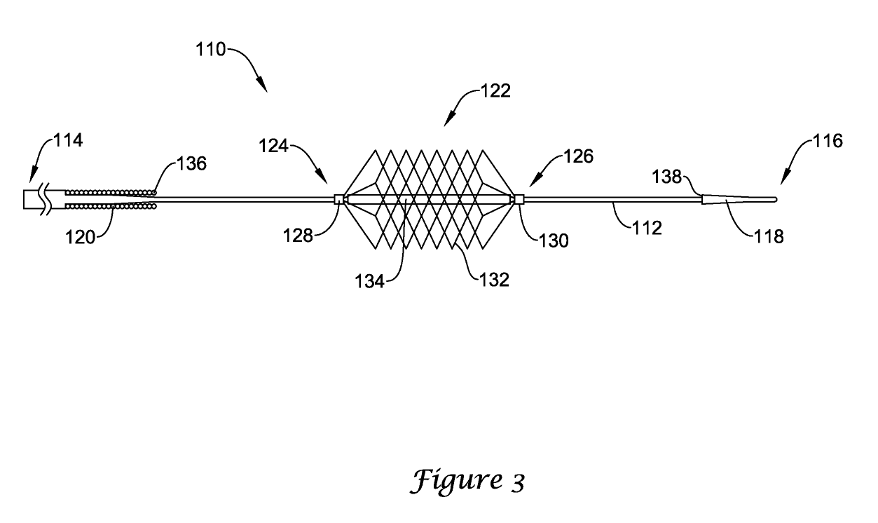

A second exemplary embodiment of a vascular recanalization device 110 is depicted in FIG. 3. The vascular recanalization device 110 is similar in many respects to the vascular recanalization device 10 of FIG. 1, with some variations. For example, the vascular recanalization device 110 may include an elongate wire 112 having a proximal end 114 and a distal end 116. The distal end 116 of the elongate wire 112 may include a distal tip, such as a distal coil tip 118 attached to the elongate wire 112, or other desired tip configuration.

The vascular recanalization device 110 may also include an expandable cage 122 coupled to the elongate wire 112. The expandable cage 122 may be similar to the expandable cage 22 discussed above regarding the vascular recanalization device 10 of FIG. 1. For instance, the expandable cage 122 may include a proximal collar 128, a distal collar 130 and an expandable mesh 132 similar in configuration and function to the proximal collar 28, distal collar 30 and expendable mesh 32, respectively, discussed above.

The expandable cage 122 may be slidably coupled to the elongate wire 112 to allow longitudinal translation of the elongate wire 112 relative to the expandable cage 122 while the expandable cage 122 remains stationary. In some embodiments the expandable cage 122 may be slidably coupled to the elongate wire 112 such that the proximal end 124 of the expandable cage 122 is slidably coupled to the elongate wire 112 and/or the distal end 126 of the expandable cage 122 is slidably coupled to the elongate wire 112. In some embodiments, no portion of the expandable cage 122 is fixedly secured to the elongate wire 112 or any other component of the vascular recanalization device 110.

As discussed above, the expandable cage 122 may include a proximal collar 128 proximate the proximal end 124 of the expandable cage 122 and a distal collar 130 proximate the distal end 126 of the expandable cage 122. The proximal collar 128 and/or the distal collar 130 may be slidably and rotatably disposed on the elongate wire 112.

The vascular recanalization device 110 may further include a central stop 134 secured to the elongate wire 112 at a location intermediate the proximal end 124 and the distal end 126 of the expandable cage 122. For example, the central stop 134 may be secured to the elongate wire 112 intermediate the proximal collar 128 and the distal collar 130 of the expandable cage 122. The expandable mesh 132 may circumferentially surround and be spaced away from the central stop 134. In some embodiments, the central stop 134 may be a helical coil member or other tubular member disposed around the elongate wire 112, or the central stop 134 may be one or more projections formed around or secured to the elongate wire 112.

The central stop 134 may be provided to prevent the expandable cage 122 from sliding distally on the elongate wire 112 distally of the central stop 134 and may be provided to prevent the expandable cage 122 from sliding proximally on the elongate wire 112 proximally of the central stop 134. For instance, the central stop 134 may prevent the proximal end 124 of the expandable cage 122 from sliding distal of the central stop 134 and the central stop 134 may prevent the distal end 126 of the expandable cage 122 from sliding proximal of the central stop 134. Thus, in the illustrative example of the vascular recanalization device 110 shown in FIG. 3, the central stop 134 may prevent the proximal collar 128 from sliding distal of the central stop 134, and the central stop 134 may prevent the distal collar 130 from sliding proximal of the central stop 134.

The vascular recanalization device 110 may also include a proximal stop 136 located proximally of the central stop 134 and a distal stop 138 located distally of the central stop 134. In some embodiments, the proximal stop 136 may be the distal end of the flexible tubular member 120 secured to the elongate wire 112 which may be similar to the flexible tubular member 20 of the vascular recanalization device 10, or the proximal stop 136 may be another component of the vascular recanalization device 110. In some embodiments, the distal stop 138 may be the proximal end of the distal coil tip 118, or the distal stop 138 may be another component of the vascular recanalization device 110.

The proximal collar 128, and thus the proximal end 124 of the expandable cage 122, may be longitudinally slidable along the elongate wire 112 between the proximal stop 136 and the central stop 134, and the distal collar 130, and thus the distal end 126 of the expandable cage 122, may be longitudinally slidable along the elongate wire 112 between the central stop 134 and the distal stop 138. Some possible distances in which the proximal collar 128 and the distal collar 130 may slide along the elongate wire 112 are disclosed above regarding the vascular recanalization device 10.

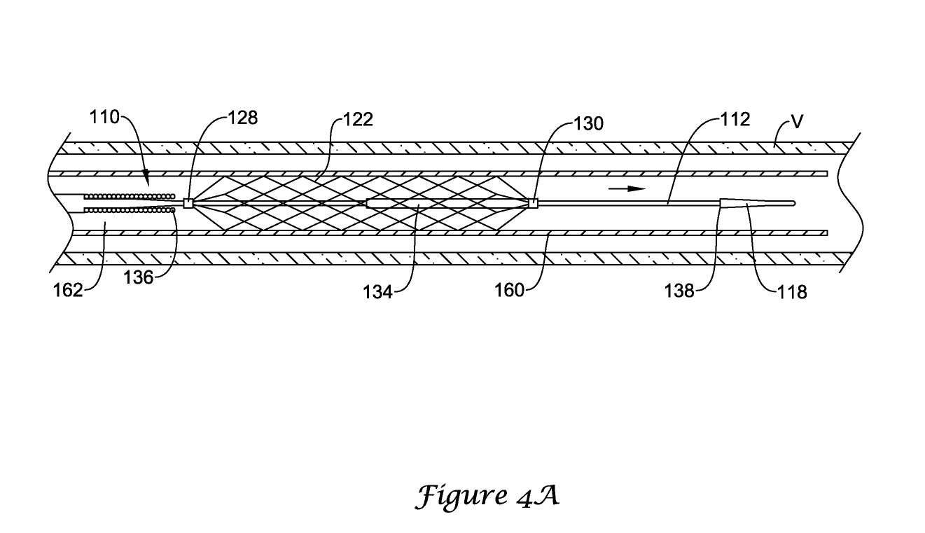

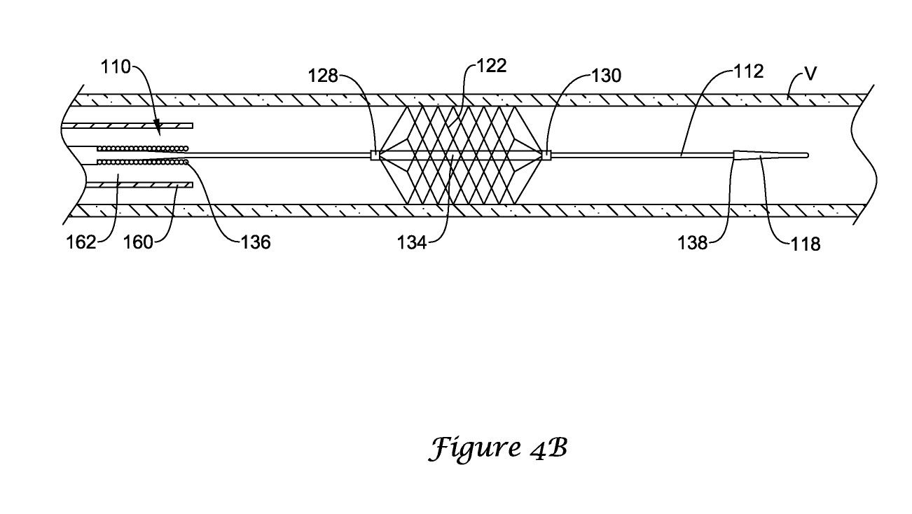

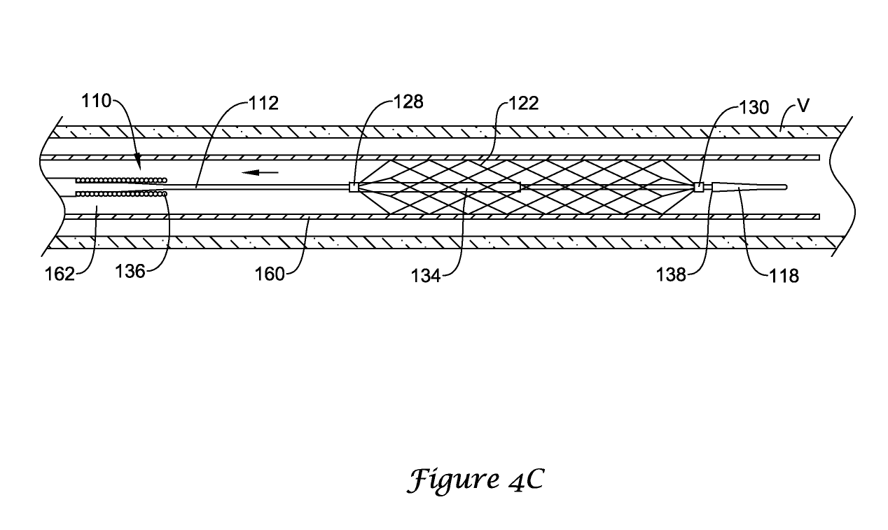

FIGS. 4A-4C illustrate an exemplary mode of operating the vascular recanalization device 110 of FIG. 3 during a medical procedure to re-establish blood flow through a vessel lumen. As shown in FIG. 4A, the vascular recanalization device 110 may be advanced distally through the lumen 162 of an elongate tubular member 160 of a medical device, such as a medical catheter (e.g., a microcatheter), during a medical procedure. For instance, the medical personnel may push the elongate wire 112 distally to advance the vascular recanalization device 110 through the elongate tubular member 160 located in a lumen of a vessel V.

While positioned in the lumen 162 of the elongate tubular member 160 the expandable cage 122 may assume a collapsed configuration in which the expandable cage 122 has a reduced diameter relative to an expanded configuration which the expandable cage 122 may be expanded to upon exiting the elongate tubular member 160. In some embodiments, the elongate tubular member 160 may constrain the expandable cage 122 to the collapsed configuration while positioned in the lumen 162 of the elongate tubular member 160. In the collapsed configuration, the expandable cage 122 may have a collapsed cage length measured from the proximal collar 128 to the distal collar 130. Furthermore, in the expanded configuration, shown in FIG. 4B, the expandable cage 122 may have an expanded cage length measured from the proximal collar 128 to the distal collar 130. The collapsed cage length may be greater than the expanded cage length.

As shown in FIG. 4A, the distance between the proximal stop 136 and the distal end of the central stop 134 may be equal to or greater than the collapsed cage length in order to allow the expandable cage 122 to be fully collapsed and elongated to the collapsed configuration while being advanced distally through the elongate tubular member 160. Furthermore, as shown in FIG. 4C, the distance between the proximal end of the central stop 134 and the distal stop 138 may be equal to or greater than the collapsed cage length in order to allow the expandable cage 122 to be fully collapsed and elongated to the collapsed configuration while being withdrawn proximally through the elongate tubular member 160.

As the elongate wire 112 is being pushed distally by the medical personnel, the central stop 134 moves distally relative to the expandable cage 122 until the distal end of the central stop 134 abuts the distal collar 130 of the expandable cage 122. Thus, the central stop 134 exerts a force on the distal collar 130, in which the exerted force has a directional component in a distal direction parallel to the longitudinal axis of the elongate wire 112, effectively resulting in the expandable cage 122 being pulled distally through the elongate tubular member 160 via the distal collar 130 by pushing the elongate wire 112 distally.

Effectively pulling the expandable cage 122 through the elongate tubular member 160 has the effect of reducing the force necessary to move the expandable cage 122 through the elongate tubular member 160 relative to situations in which the expandable cage 122 would be pushed through the elongate tubular member 160. Pulling the expandable cage 122 distally from the distal collar 130 will tend to cause the expandable cage 122 to lengthen and thus tend to decrease in diameter, reducing frictional forces between the expandable cage 122 and the inner surface of the elongate tubular member 160, whereas pushing the expandable cage 122 distally from the proximal collar 128 would tend to cause the expandable cage 122 to shorten and thus tend to enlarge in diameter, increasing frictional forces between the expandable cage 122 and the inner surface of the elongate tubular member 160.

The vascular recanalization device 110 may be advanced distally until the expandable cage 122 is deployed from the distal end of the elongate tubular member 160 to deliver the expandable cage 122 to a target location within the vessel V. As shown in FIG. 4B, once deployed from the elongate tubular member 160, the expandable cage 122 may be expanded to the expanded configuration in which the expandable cage 122 is expanded in diameter and shortened in length. In some embodiments, the expandable cage 122 may be self-expanding in which the expandable cage 122 automatically expands upon being deployed from the elongate tubular member 160. In other embodiments, the expandable cage 122 may be manually, selectively and/or controllably expanded, such as by the manipulation of a component of the vascular recanalization device 110, application of a stimulus, and/or activation/deactivation of an energy source, for example. In the expanded configuration, the expandable cage 122 may exert a radially outward force on the vessel V to open a blockage and re-establish blood flow through the vessel V. The openness of the interstitial openings between filaments of the expandable mesh 132 allows blood to flow through the expandable cage 122. Furthermore, in some embodiments, the expandable mesh 132 may be configured to capture embolic material while deployed in the vessel V.

When the expandable cage 122 is in its expanded configuration and deployed in the vessel V, expandable cage 122 may be shortened in length such that the central stop 134 abuts both the proximal collar 128 and the distal collar 130, simultaneously. Thus, the central stop 134 may prevent further shortening and/or radial expansion of the expandable cage 122 beyond a threshold amount.

As shown in FIG. 4C, the vascular recanalization device 110 may be withdrawn proximally through the lumen 162 of an elongate tubular member 160 of a medical device, such as a medical catheter (e.g., a microcatheter), at the completion of the medical procedure. For instance, the medical personnel may pull the elongate wire 112 proximally to withdraw the vascular recanalization device 110 through the elongate tubular member 160.

As the elongate wire 112 is being pulled proximally by the medical personnel, the central stop 134 moves proximally relative to the expandable cage 122 until the proximal end of the central stop 134 abuts the proximal collar 128 of the expandable cage 122. Thus, the central stop 134 exerts a force on the proximal collar 128, in which the exerted force has a directional component in a proximal direction parallel to the longitudinal axis of the elongate wire 112, effectively resulting in the expandable cage 122 being pulled proximally through the elongate tubular member 160 via the proximal collar 128 by pulling the elongate wire 112 proximally.

Effectively pulling the expandable cage 122 through the elongate tubular member 160 has the effect of reducing the force necessary to move the expandable cage 122 through the elongate tubular member 160 relative to situations in which the expandable cage 122 would be pushed through the elongate tubular member 160. Pulling the expandable cage 122 proximally from the proximal collar 128 will tend to cause the expandable cage 122 to lengthen and thus tend to decrease in diameter, reducing frictional forces between the expandable cage 122 and the inner surface of the elongate tubular member 160, whereas pushing the expandable cage 122 proximally from the distal collar 130 would tend to cause the expandable cage 122 to shorten and thus tend to enlarge in diameter, increasing frictional forces between the expandable cage 122 and the inner surface of the elongate tubular member 160.

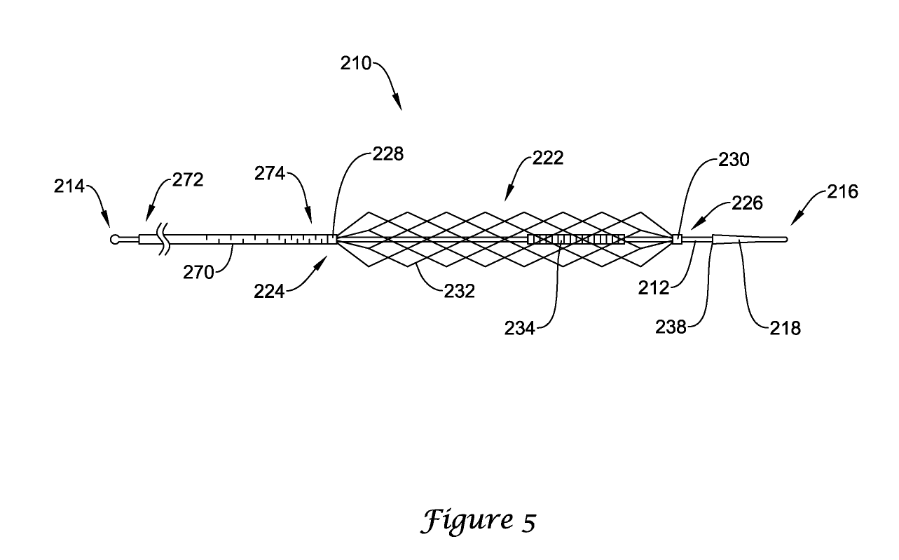

A third illustrative embodiment of a vascular recanalization device 210 is depicted in FIG. 5. The vascular recanalization device 210 may include an elongate wire 212 having a proximal end 214 and a distal end 216. In operation, the distal end 216 of the elongate wire 212 may be advanced through the vasculature of a patient while the proximal end 214 remains exterior of the patient to be manipulated by the medical personnel during a medical procedure.

The distal end 216 of the elongate wire 212 may include a distal tip, such as a distal coil tip 218 attached to the elongate wire 212. For example, the distal coil tip 218 may include a wire filament helically wound into a coil. The coil may be disposed over a distal portion of the elongate wire 212 and secured to the elongate wire 212, such as by welding, soldering, brazing, or adhesive bonding in some instances. In other embodiments, the distal tip of the elongate wire 212 may have a different configuration, if desired.

The vascular recanalization device 210 may also include an expandable cage 222 coupled to the elongate wire 212. The expandable cage 222 may be similar to the expandable cage 22 discussed above regarding the vascular recanalization device 10 of

FIG. 1. For instance, the expandable cage 222 may include a proximal collar 228, a distal collar 230 and an expandable mesh 232 similar in configuration and function to the proximal collar 28, distal collar 30 and expendable mesh 32, respectively, discussed above.

The expandable cage 222 may be slidably coupled to the elongate wire 212 to allow longitudinal translation of the elongate wire 212 relative to the expandable cage 222 while the expandable cage 222 remains stationary. In some embodiments the expandable cage 222 may be slidably coupled to the elongate wire 212 such that the proximal end 224 of the expandable cage 222 is slidably coupled to the elongate wire 212 and/or the distal end 226 of the expandable cage 222 is slidably coupled to the elongate wire 212. In some embodiments, no portion of the expandable cage 222 is fixedly secured to the elongate wire 212 or any other component of the vascular recanalization device 210.

As discussed above, the expandable cage 222 may include a proximal collar 228 proximate the proximal end 224 of the expandable cage 222 and a distal collar 230 proximate the distal end 226 of the expandable cage 222. The proximal collar 228 and/or the distal collar 230 may be slidably and rotatably disposed on the elongate wire 212.

The vascular recanalization device 210 may also include an elongate tubular member 270 disposed over the elongate wire 212 such that the elongate wire 212 is longitudinally movable through the elongate tubular member 270. The elongate tubular member 270 may have any desired length such that a proximal end 272 of the elongate tubular member 270 may be located exterior of a patient during a medical procedure while a distal end 274 of the elongate tubular member 270 may be located within the vessel of the patient and proximal of the expandable cage 222 during the medical procedure. In some embodiments, the elongate tubular member 270 may be disconnected from the expandable cage 222 such that the expandable cage 222 is longitudinally moveable relative to the elongate wire 212 while the elongate tubular member 270 remains stationary. In the embodiment illustrated in FIG. 5, however, the proximal end 224 of the expandable cage 222 is attached to the distal end 274 of the elongate tubular member 270. For instance, the elongate tubular member 270 may be attached to the proximal collar 228 of the expandable cage 222. In some embodiments, the proximal collar 228 may be, at least in part, formed of a portion of the elongate tubular member 270.

The vascular recanalization device 210 may further include a central stop 234 secured to the elongate wire 212 at a location intermediate the proximal end 224 and the distal end 226 of the expandable cage 222. For example, the central stop 234 may be secured to the elongate wire 212 intermediate the proximal collar 228 and the distal collar 230 of the expandable cage 222. The expandable mesh 232 may circumferentially surround and be spaced away from the central stop 234. In some embodiments, the central stop 234 may be a helical coil member or other tubular member disposed around the elongate wire 212, or the central stop 234 may be one or more projections formed around or secured to the elongate wire 212.

The central stop 234 may be provided to prevent the expandable cage 222 from sliding distally on the elongate wire 212 distally of the central stop 234 and may be provided to prevent the expandable cage 222 from sliding proximally on the elongate wire 212 proximally of the central stop 234. For instance, the central stop 234 may prevent the proximal end 224 of the expandable cage 222 from sliding distal of the central stop 234 and the central stop 234 may prevent the distal end 226 of the expandable cage 222 from sliding proximal of the central stop 234. Thus, in the illustrative example of the vascular recanalization device 210 shown in FIG. 5, the central stop 234 may prevent the proximal collar 228 from sliding distal of the central stop 234, and the central stop 234 may prevent the distal collar 230 from sliding proximal of the central stop 234.

Furthermore, in some embodiments the central stop 234 may function to limit shortening of the expandable cage 222 beyond a threshold amount when the expandable cage 222 is deployed in a vessel lumen. For instance, in a fully expanded configuration, the central stop 234 may prevent further relative movement of the proximal collar 228 toward the distal collar 230 of the expandable cage 222.

The vascular recanalization device 210 may also include a distal stop 238 located distally of the central stop 234. In some embodiments, the distal stop 238 may be the proximal end of the distal coil tip 218, or the distal stop 238 may be another component of the vascular recanalization device 210. The distal collar 230, and thus the distal end 226 of the expandable cage 222, may be longitudinally slidable along the elongate wire 212 between the central stop 234 and the distal stop 238.

In some embodiments, for example in embodiments in which the expandable cage 222 is not attached to the elongate tubular member 270, the vascular recanalization device 210 may also include a proximal stop located proximally of the central stop 234. In some embodiments, the proximal stop may be the distal end 274 of the elongate tubular member 270, or the proximal stop may be another component of the vascular recanalization device 210. In embodiments in which the proximal stop is the distal end 274 of the elongate tubular member 270, the proximal stop may be longitudinally displaceable relative to the central stop 234. In such embodiments, the proximal collar 228, and thus the proximal end 224 of the expandable cage 222, may be longitudinally slidable along the elongate wire 212 between the proximal stop and the central stop 234.

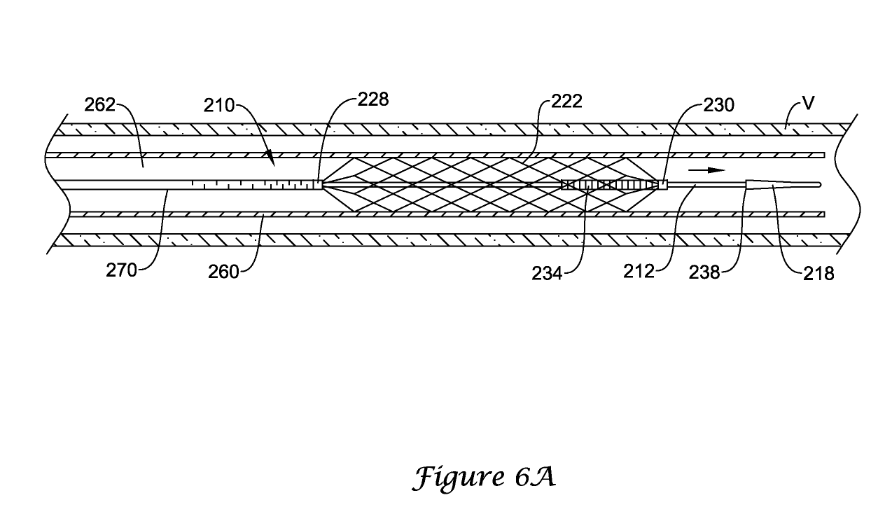

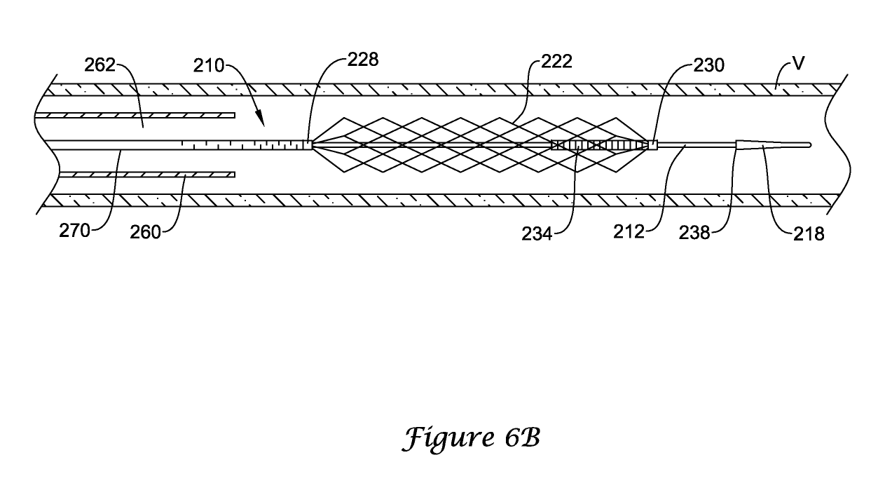

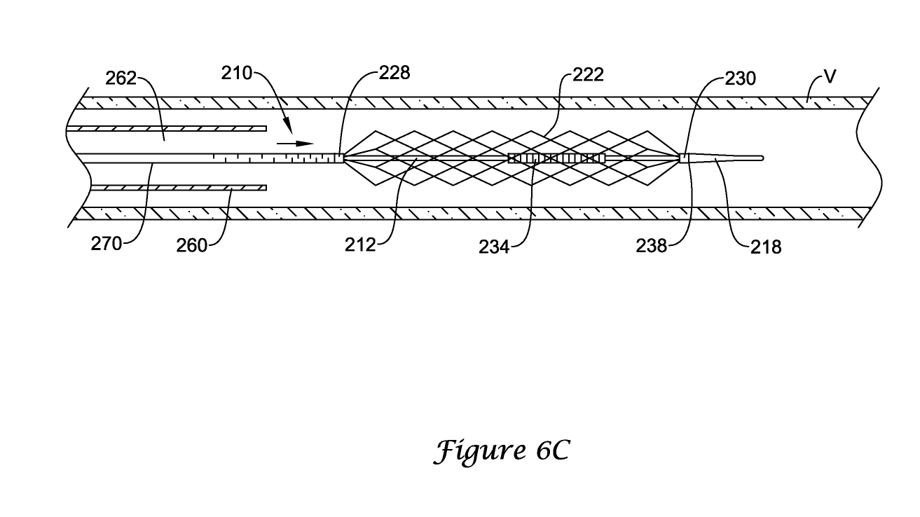

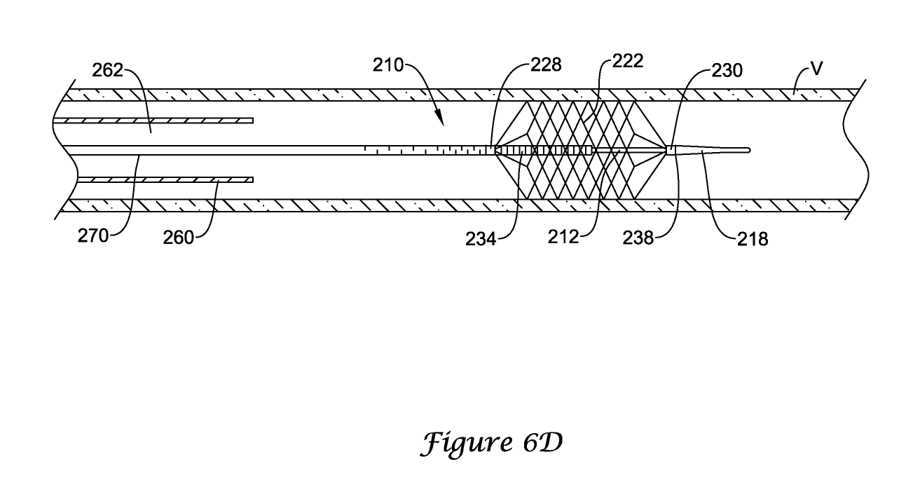

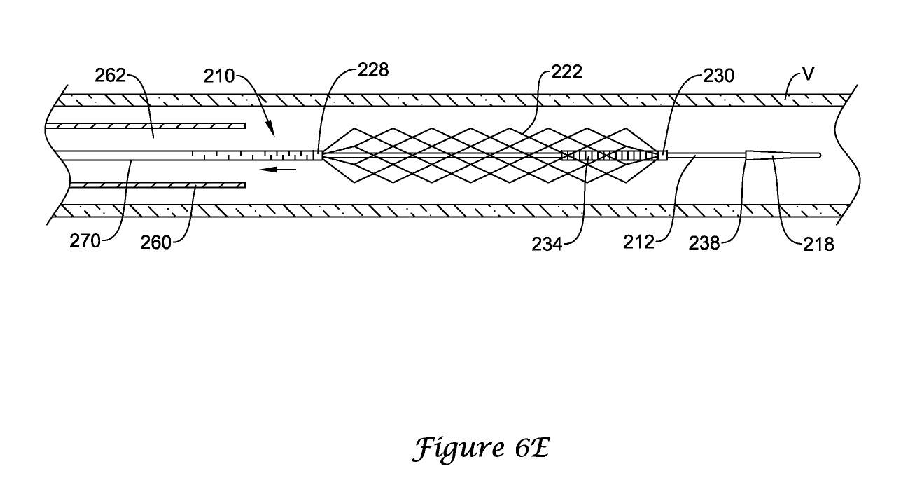

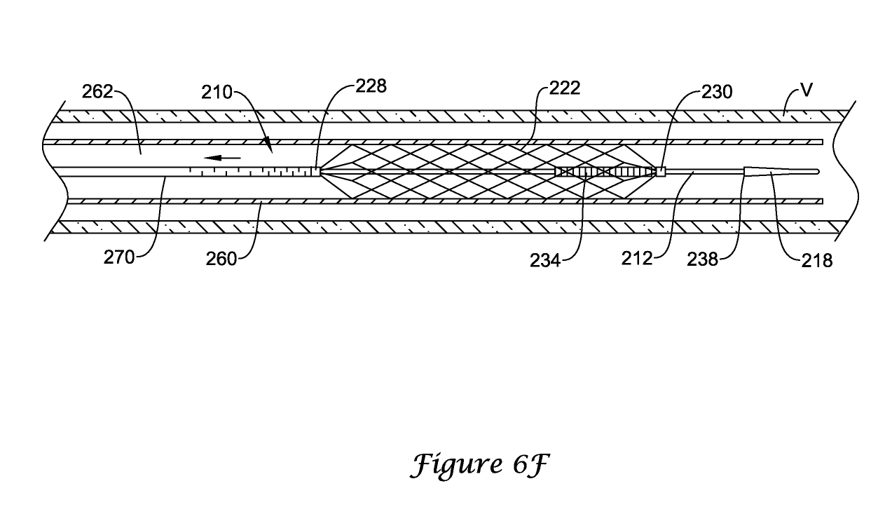

FIGS. 6A-6F illustrate an exemplary mode of operating the vascular recanalization device 210 of FIG. 5 during a medical procedure to re-establish blood flow through a vessel lumen. As shown in FIG. 6A, the vascular recanalization device 210 may be advanced distally through the lumen 262 of an elongate tubular member 260 of a medical device, such as a medical catheter (e.g., a microcatheter), during a medical procedure. For instance, the medical personnel may simultaneously push the elongate wire 212 and elongate tubular member 270 distally to advance the vascular recanalization device 210 through the elongate tubular member 260 located in a lumen of a vessel V.

While positioned in the lumen 262 of the elongate tubular member 260 the expandable cage 222 may assume a collapsed configuration in which the expandable cage 222 has a reduced diameter relative to an expanded configuration which the expandable cage 222 may be expanded to upon exiting the elongate tubular member 260. In some embodiments, the elongate tubular member 260 may constrain the expandable cage 222 to the collapsed configuration while positioned in the lumen 262 of the elongate tubular member 260. In the collapsed configuration, the expandable cage 222 may have a collapsed cage length measured from the proximal collar 228 to the distal collar 230. Furthermore, in the expanded configuration, shown in FIG. 6D, the expandable cage 222 may have an expanded cage length measured from the proximal collar 228 to the distal collar 230. The collapsed cage length may be greater than the expanded cage length.

As shown in FIG. 6A, the distance between the proximal collar 228 and the distal collar in the collapsed configuration may be maintained by controlling the distance between the distal end 274 of the elongate tubular member 270 and the distal end of the central stop 234, effectively stretching the expandable cage 222 between the distal end 274 of the elongate tubular member 270 and the distal end of the central stop 234 secured to the elongate wire 212.

As the vascular recanalization device 210 is being pushed distally by the medical personnel, the central stop 234 may abut the distal collar 230 of the expandable cage 222. Thus, the central stop 234 exerts a force on the distal collar 230, in which the exerted force has a directional component in a distal direction parallel to the longitudinal axis of the elongate wire 212, effectively resulting in the expandable cage 222 being pulled distally through the elongate tubular member 260 via the distal collar 230 by pushing the elongate wire 212 distally.

Effectively pulling the expandable cage 222 through the elongate tubular member 260 has the effect of reducing the force necessary to move the expandable cage 222 through the elongate tubular member 260 relative to situations in which the expandable cage 222 would be pushed through the elongate tubular member 260. Pulling the expandable cage 222 distally from the distal collar 230 will tend to cause the expandable cage 222 to lengthen and thus tend to decrease in diameter, reducing frictional forces between the expandable cage 222 and the inner surface of the elongate tubular member 260, whereas pushing the expandable cage 222 distally from the proximal collar 228 would tend to cause the expandable cage 222 to shorten and thus tend to enlarge in diameter, increasing frictional forces between the expandable cage 222 and the inner surface of the elongate tubular member 260.

The vascular recanalization device 210 may be advanced distally until the expandable cage 222 is deployed from the distal end of the elongate tubular member 260, shown in FIG. 6B, to deliver the expandable cage 222 to a target location within the vessel V. Once deployed from the elongate tubular member 260, the expandable cage 222 may be expanded to the expanded configuration in which the expandable cage 222 is expanded in diameter and shortened in length. In some embodiments, the expandable cage 222 may be self-expanding in which the expandable cage 222 automatically expands upon being deployed from the elongate tubular member 260. In other embodiments, the expandable cage 222 may be manually, selectively and/or controllably expanded, such as by the manipulation of a component of the vascular recanalization device 210, application of a stimulus, and/or activation/deactivation of an energy source, for example. In some embodiments, the expandable cage 222 may be partially self-expanding in which the expandable cage 222 partially expands automatically followed by further manual, selective and/or controlled expansion to the expanded configuration. In the expanded configuration, the expandable cage 222 may exert a radially outward force on the vessel V to open a blockage and re-establish blood flow through the vessel V. The openness of the interstitial openings between filaments of the expandable mesh 232 allows blood to flow through the expandable cage 222. Furthermore, in some embodiments, the expandable mesh 232 may be configured to capture embolic material while deployed in the vessel V.

As illustrated in FIGS. 6C and 6D, once deployed from the elongate tubular member 260, the expandable cage 222 may be manually expanded from a collapsed configuration shown in FIG. 6B to a fully expanded configuration shown in FIG. 6D by pulling the elongate wire 212 proximally relative to the elongate tubular member 270 of the vascular recanalization device 210 and/or by pushing the elongate tubular member 270 distally relative to the elongate wire 212 of the vascular recanalization device 210. In some embodiments, the expandable cage 222 may automatically partially expand upon deployment from the elongate tubular member 260, and may then be further expanded to a fully expanded configuration by pulling the elongate wire 212 proximally relative to the elongate tubular member 270 and/or pushing the elongate tubular member 270 distally relative to the elongate wire 212.

When the expandable cage 222 is deployed in the vessel V, the elongate wire 212 may be longitudinally translated through the expandable cage 222 and/or rotated while the expandable cage 222 stays stationary, allowing the expandable cage 222 to float on the elongate wire 212 up to a threshold amount. Thus, the expandable cage 222 may float on the elongate wire 212 as the proximal collar 228 slides along the elongate wire 212 proximal of the central stop 234, and the distal collar 230 slides along the elongate wire 212 between the central stop 234 and the distal stop 238.

For instance, once deployed from the elongate tubular member 260, the elongate wire 212 is free to move longitudinally between a first position in which the distal collar 230 abuts the central stop 234 and is spaced away from the distal stop 238 and a second position in which the distal collar 230 abuts the distal stop 238 and is spaced away from the central stop 234. Thus, the elongate wire 212 is free to move longitudinally a distance equal to the distance between the central stop 234 and the distal stop 238 minus the length of the distal collar 230. This distance may be considered the float length of the expandable cage 222 on the elongate wire 212. In some embodiments, the float length may be 1 mm or more, 2 mm or more, 3 mm or more, 4 mm or more, 5 mm or more, 6 mm or more, 7 mm or more, 8 mm or more, 9 mm or more, 10 mm or more, 15 mm or more, 20 mm or more, or 30 mm or more, for example.

The length of the central stop 234 may be any desired length. In some embodiments, the length of the central stop 234 may be 5% or less, 10% or less, 15% or less, 20% or less, 25% or less, 50% or less, 75% or less, 80% or less, 85% or less, 90% or less, or 95% or less of the distance between the proximal collar 228 and the distal collar 230 when the expandable cage 222 is in the collapsed configuration. In some embodiments, the length of the central stop 234 may be 5% or more, 10% or more, 15% or more, 20% or more, 25% or more, 50% or more, 75% or more, 80% or more, 85% or more, 90% or more, or 95% or more of the distance between the proximal collar 228 and the distal collar 230 when the expandable cage 222 is in the collapsed configuration. In some embodiments, the central stop 234 may have a length of about 1 mm, about 2 mm, about 3 mm, about 4 mm, or about 5 mm, for example.

As the elongate wire 212 is pulled proximally and/or the elongate tubular member 270 is pushed distally, the distal stop 238 may abut the distal collar 230 of the expandable cage 222, as shown in FIG. 6C. Further proximal movement of the elongate wire 212 relative to the elongate tubular member 270 and/or distal movement of the elongate tubular member 270 relative to the elongate wire 212 will result in the expandable cage 222 shortening in length as the distal collar 230 is moved toward the proximal collar 228. The distal collar 230 may be drawn toward the proximal collar 228 until the central stop 234 abuts the proximal collar 228, sandwiching the proximal collar 228 between the distal end 274 of the elongate tubular member 270 and the central stop 234, shown in FIG. 6D. In this configuration, the expandable cage 222 is fully expanded to its expanded configuration in which the expandable cage 222 is expanded in diameter and shortened in length. In the expanded configuration, the expandable cage 222 may exert a radially outward force on the vessel V to open a blockage and re-establish blood flow through the vessel V.

In order to return the expandable cage 222 to its collapsed configuration for withdrawal from the vessel V, the proximal collar 228 may be moved away from the distal collar 230, lengthening the expandable cage 222. To this end, the elongate tubular member 270 may be moved proximally relative to the elongate wire 212 and/or the elongate wire 212 may be moved distally relative to the elongate tubular member 270, as shown in FIG. 6E. Relative movement between the elongate tubular member 270 and the elongate wire 212 results in stretching the expandable cage 222 between the distal end 274 of the elongate tubular member 270 and the distal end of the central stop 234.

As shown in FIG. 6F, the vascular recanalization device 210 may be withdrawn proximally through the lumen 262 of an elongate tubular member 260 of a medical device, such as a medical catheter (e.g., a microcatheter), at the completion of the medical procedure. For instance, the medical personnel may pull the elongate wire 212 and elongate tubular member 270 proximally to withdraw the vascular recanalization device 210 through the elongate tubular member 260. Prior to withdrawing the expandable cage 222 into the elongate tubular member 260, the central stop 234 may be moved away from the distal end 274 of the elongate tubular member 270 and/or the proximal collar 228 (e.g., by moving the elongate wire 212 distally relative to the elongate tubular member 270 and/or moving the elongate tubular member 270 proximally relative to the elongate wire 212) to allow the collars 228, 230 of the expandable cage 222 to move away from one another so the expandable cage 222 can be returned to a collapsed configuration.

Pulling the expandable cage 222 proximally into the elongate tubular member 260 may cause the expandable cage 222 to further elongate such that the distal collar 230 moves out of contact with the central stop 234, as shown in FIG. 6F.

As the vascular recanalization device 210 is being pulled proximally by the medical personnel, pulling of the elongate tubular member 270 proximally exerts a force on the proximal collar 228, in which the exerted force has a directional component in a proximal direction parallel to the longitudinal axis of the elongate wire 212, effectively resulting in the expandable cage 222 being pulled proximally through the elongate tubular member 260 via the proximal collar 228 by pulling the elongate tubular member 270 proximally.

Effectively pulling the expandable cage 222 through the elongate tubular member 260 has the effect of reducing the force necessary to move the expandable cage 222 through the elongate tubular member 260 relative to situations in which the expandable cage 222 would be pushed through the elongate tubular member 260. Pulling the expandable cage 222 proximally from the proximal collar 228 will tend to cause the expandable cage 222 to lengthen and thus tend to decrease in diameter, reducing frictional forces between the expandable cage 222 and the inner surface of the elongate tubular member 260, whereas pushing the expandable cage 222 proximally from the distal collar 230 would tend to cause the expandable cage 222 to shorten and thus tend to enlarge in diameter, increasing frictional forces between the expandable cage 222 and the inner surface of the elongate tubular member 260.

In an alternative embodiment in which the distal end 274 of the elongate tubular member 270 is not attached to the proximal collar 228 of the expandable cage 222, during advancement of the vascular recanalization device 210 distally through the elongate tubular member 260, the proximal stop (e.g., the distal end 274 of the elongate tubular member 270) may be located proximal of the central stop 234 a distance which will allow the expandable cage 222 to be fully collapsed and elongated to the collapsed configuration while being advanced distally through the elongate tubular member 260. In other words, the distance between the proximal stop and the distal end of the central stop 234 may be greater than the distance between the proximal collar 228 and the distal collar 230 in the collapsed configuration. Upon deployment of the expandable cage 222 in a vessel V, the proximal stop may be moved toward the distal stop 238 (e.g., by moving the elongate wire 212 proximally relative to the elongate tubular member 270 and/or moving the elongate tubular member 270 distally relative to the elongate wire 212). In moving the proximal stop toward the distal stop 238, the proximal stop contacts the proximal collar 228 and the distal stop 238 contacts the distal collar 230. Further movement draws the distal collar 230 toward the proximal collar 228, shortening the length of the expandable cage 222 in order to expand the expandable cage 222 to its expanded configuration. In the expanded configuration, the expandable cage 222 may exert a radially outward force on the vessel V to open a blockage and re-establish blood flow through the vessel V.

When the expandable cage 222 is in its expanded configuration and deployed in the vessel V, the elongate wire 212 may be longitudinally translated through the expandable cage 222 and/or rotated while the expandable cage 222 stays stationary and engaged against the vessel V, allowing the expandable cage 222 to float on the elongate wire 212. Thus, longitudinal movement of the elongate wire 212 up to a threshold amount, whether inadvertent or intentional, will not cause the expandable cage 222 to move.

For instance, in the expanded configuration, the elongate wire 212 is free to move longitudinally between a first position in which the central stop 234 abuts the distal collar 230 and is spaced away from the proximal collar 228 and a second position in which the central stop 234 abuts the proximal collar 228 and is spaced away from the distal collar 230. Thus, the elongate wire 212 is free to move longitudinally a distance equal to the distance between the proximal collar 228 and the distal collar 230 minus the length of the central stop 234. This distance may be considered the float length of the expandable cage 222 on the elongate wire 212. In some embodiments, the float length may be 1 mm or more, 2 mm or more, 3 mm or more, 4 mm or more, 5 mm or more, 6 mm or more, 7 mm or more, 8 mm or more, 9 mm or more, 10 mm or more, 15 mm or more, 20 mm or more, or 30 mm or more, for example.

The distal collar 230 may be drawn toward the proximal collar 228 up to a threshold amount until the central stop 234 abuts both the proximal collar 228 and the distal collar 230, sandwiching the central stop 234 between the proximal collar 228 and the distal collar 230 and restricting the expandable cage 222 from floating on the elongate wire 212. In some embodiments, the length of the central stop 234 may be chosen to control the extent of expansion of the expandable cage 222.

In order to return the expandable cage 222 to its collapsed configuration for withdrawal from the vessel V, the proximal collar 228 may be moved away from the distal collar 230, lengthening the expandable cage 222. To this end, the elongate tubular member 270 may be moved proximally relative to the elongate wire 212 and/or the elongate wire 212 may be moved distally relative to the elongate tubular member 270 to increase the distance between the proximal stop and the distal stop 238. The distance between the proximal stop and the distal stop 238 may be equal to or greater than the collapsed cage length in order to allow the expandable cage 222 to be fully collapsed and elongated to the collapsed configuration while being withdrawn proximally through the elongate tubular member 260.

The vascular recanalization device 210 may then be withdrawn into the elongate tubular member 260. For instance, when pulling the vascular recanalization device 210 proximally, the distal stop 238 may contact the distal collar 230 of the expandable cage 222, pushing the expandable cage 222 into the elongate tubular member 260.

FIGS. 7-10 illustrate various exemplary embodiments of an expandable mesh which may be used in the expandable cage of the vascular recanalization devices shown in FIGS. 1, 3 and 5. It is noted that the various embodiments shown in FIGS. 7-10 are illustrated as if the tubular construct of the expandable mesh were cut longitudinally and then unrolled into a flattened configuration to better illustrate the mesh patterns.

A first exemplary embodiment of an expandable mesh 332, shown in FIG. 7, includes a repeating pattern of interconnected filaments 348 defining interstitial openings 352 defined between adjacent filaments 348. The pattern, as well as other patterns of an expandable mesh described herein, may be manufactured by interweaving or interconnecting a plurality of individual fibers to form a tubular construct, the pattern may be manufactured from removing portions of a tubular member, or the pattern may be manufactured from removing portions of a flat sheet and then rolled into a tubular construct, and then incorporated into the expandable cage 22, for example. The expandable mesh 332 may include end filaments or extensions 350 extending from the main meshwork. The extensions 350 may be configured to be coupled to the collars 28, 30 of the expandable cage 22.

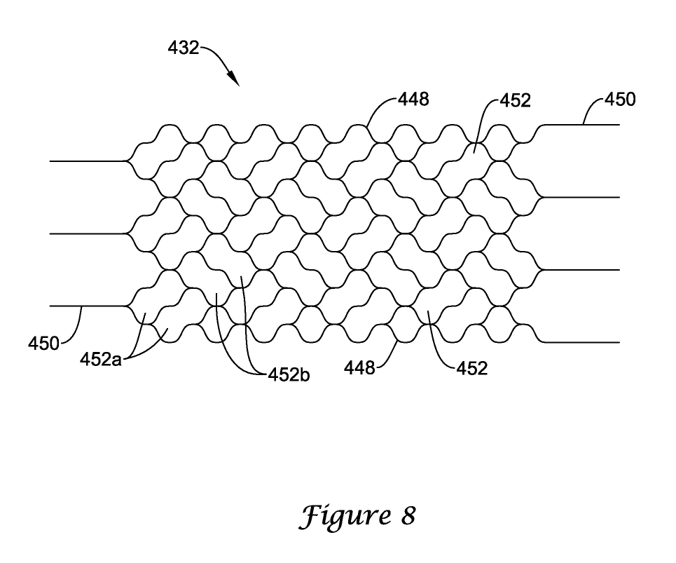

FIG. 8 illustrates a second exemplary embodiment of an expandable mesh 432. The expandable mesh 432 includes a repeating pattern of interconnected filaments 448 defining interstitial openings 452 defined between adjacent filaments 448. The interstitial openings 452 may resemble a peanut shape, having two enlarged end regions spaced apart by a narrower central region. The pattern may include a pair of adjoining interstitial openings 452a extending in a first direction adjacent a pair of adjoining interstitial openings 452b extending in a second direction, generally orthogonal to the first direction. This arrangement of adjacent pairs of interstitial openings 452 may be repeated throughout the expandable mesh 432 pattern. The expandable mesh 432 may include end filaments or extensions 450 extending from the main meshwork, which may be configured to be coupled to the collars 28, 30 of the expandable cage 22.

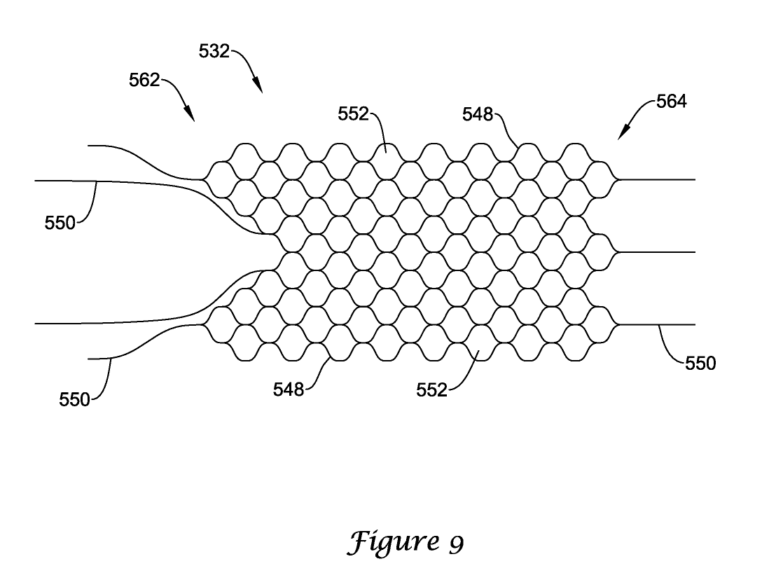

FIG. 9 illustrates another exemplary embodiment of an expandable mesh 532, including a repeating pattern of interconnected filaments 548 defining interstitial openings 552 defined between adjacent filaments 548. The expandable mesh 532 may include end filaments or extensions 550 extending from the main meshwork, which may be configured to be coupled to the collars 28, 30 of the expandable cage 22. As shown in FIG. 9, the proximal end 562 of the expandable mesh 532 may be configured differently than the distal end 564 of the expandable mesh 532. It can be appreciated that as the expandable mesh 532 is rolled into a tubular construct, the distal end 564 of the expandable mesh 532 may extend radially inward toward the central longitudinal axis of the expandable mesh 532, forming a closed distal end, whereas the proximal end 562 of the expandable mesh 532 may create a proximal mouth or opening. Such a configuration may allow embolic material to flow into the proximal mouth and be trapped within the expandable mesh 532.

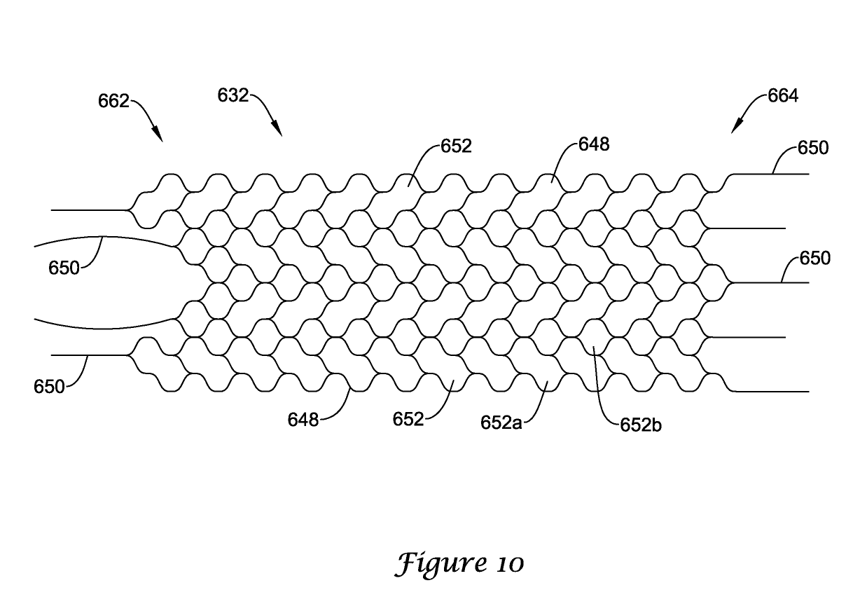

FIG. 10 illustrates yet another exemplary embodiment of an expandable mesh 632 including a repeating pattern of interconnected filaments 648 defining interstitial openings 652 defined between adjacent filaments 648. The expandable mesh 632 may include end filaments or extensions 650 extending from the main meshwork, which may be configured to be coupled to the collars 28, 30 of the expandable cage 22. The pattern may include a longitudinal row of large interstitial openings 652a followed by a longitudinal row of small interstitial openings 652b. Rows of large interstitial openings 652a and rows of small intersitical openings 652b may be alternatingly arranged around the circumference of the expandable mesh 632. Similar to the embodiment of FIG. 9, when the expandable mesh 632 is rolled into a tubular construct, the distal end 664 of the expandable mesh 632 may extend radially inward toward the central longitudinal axis of the expandable mesh 632, forming a closed distal end, whereas the proximal end 662 of the expandable mesh 632 may create a proximal mouth or opening. Such a configuration may allow embolic material to flow into the proximal mouth and be trapped within the expandable mesh 632.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A medical device comprising:
an elongate wire having a proximal end and a distal end;
an expandable cage having a proximal end and a distal end, the expandable cage being coupled to the elongate wire such that the expandable cage is longitudinally slidable along a length of the elongate wire between a first position and a second position; and
at least one stop secured to the elongate wire between the proximal end and the distal end of the expandable cage;
wherein the proximal end of the expandable cage is prevented from sliding distally beyond the at least one stop and the distal end of the expandable cage is prevented from sliding proximally beyond the at least one stop.

2. The medical device of claim 1, wherein the proximal end of the expandable cage is slidably coupled to the elongate wire and the distal end of the expandable cage is slidably coupled to the elongate wire.

3. The medical device of claim 2, wherein the elongate wire is longitudinally translatable through the expandable cage while the cage remains stationary.

4. The medical device of claim 1, wherein the expandable cage includes a proximal collar slidably disposed on the elongate wire, a distal collar slidably disposed on the elongate wire, and an expandable mesh extending between the proximal collar and the distal collar.

5. The medical device of claim 4, wherein the at least one stop is located between the proximal collar and the distal collar of the expandable cage.

6. The medical device of claim 5, wherein the expandable mesh circumferentially surrounds and is spaced away from the at least one stop.

7. The medical device of claim 5, further comprising a proximal stop located proximal of the proximal collar and a distal stop located distal of the distal collar.

8. The medical device of claim 7, wherein the proximal collar is slidable along the elongate wire between the proximal stop and the at least one stop, and the distal collar is slidable along the elongate wire between the at least one stop and the distal stop.

9. The medical device of claim 8, wherein in the first position the at least one stop abuts the distal collar and is spaced away from the proximal collar, and in the second position the at least one stop abuts the proximal collar and is spaced away from the distal collar.

10. The medical device of claim 7, wherein the expandable cage has an expanded cage length and a collapsed cage length, wherein the distance between the proximal stop and a distal end of the at least one stop is equal to or greater than the collapsed cage length, and wherein the distance between the distal stop and a proximal end of the at least one stop is equal to or greater than the collapsed cage length.

11. A vascular recanalization device comprising:
an elongate wire having a proximal end and a distal end;
an expandable cage slidably coupled to the elongate wire, the expandable cage including a proximal collar slidably disposed on the elongate wire and a distal collar slidably disposed on the elongate wire;
a central stop secured to the elongate wire intermediate the proximal collar and the distal collar;
a proximal stop located proximal of the proximal collar of the expandable cage; and
a distal stop located distal of the distal collar of the expandable cage;
wherein the proximal collar is slidable along the elongate wire between the proximal stop and the central stop, and the distal collar is slidable along the elongate wire between the central stop and the distal stop.

12. The vascular recanalization device of claim 11, wherein the expandable cage has an expanded cage length and a collapsed cage length, wherein the distance between the proximal stop and a distal end of the central stop is equal to or greater than the collapsed cage length, and wherein the distance between the distal stop and a proximal end of the central stop is equal to or greater than the collapsed cage length.

13. The vascular recanalization device of claim 11, wherein in a first position the central stop abuts the distal collar and is spaced away from the proximal collar, and in a second position the central stop abuts the proximal collar and is spaced away from the distal collar.

14. The vascular recanalization device of claim 11, wherein the expandable cage includes an expandable mesh extending between the proximal collar and the distal collar, the expandable mesh circumferentially surrounding and spaced away from the central stop.

15. The vascular recanalization device of claim 11, wherein the proximal stop is longitudinally displaceable relative to the central stop.

16. The vascular recanalization device of claim 11, wherein the proximal stop is the distal end of an elongate tubular member slidably disposed over the elongate wire.

17. A method of treating a vessel lumen, the method comprising:
providing a medical device including:
an elongate wire having a proximal end and a distal end;
an expandable cage slidably coupled to the elongate wire, the expandable cage including a proximal collar slidably disposed on the elongate wire and a distal collar slidably disposed on the elongate wire; and
a central stop secured to the elongate wire intermediate the proximal collar and the distal collar;
pushing the elongate wire distally, whereby the central stop abuts the distal collar of the expandable cage and is spaced away from the proximal collar; and
pulling the elongate wire proximally, whereby the central stop abuts the proximal collar of the expandable cage and is spaced away from the distal collar.

18. The method of claim 17, wherein during the step of pushing the elongate wire distally, the expandable cage is advanced distally through an elongate tubular member.

19. The method of claim 18, wherein the expandable cage is pulled distally through the elongate tubular member by pushing the elongate wire distally.

20. The method of claim 17, wherein during the step of pulling the elongate wire proximally, the expandable cage is withdrawn proximally through an elongate tubular member.

21. The method of claim 20, wherein the expandable cage is pulled proximally through the elongate tubular member by pulling the elongate wire proximally.

22. The method of claim 17, wherein during the step of pushing the elongate wire distally, the central stop exerts a force on the distal collar, wherein the force has a directional component in a distal direction parallel to a longitudinal axis of the elongate wire.

23. The method of claim 17, wherein during the step of pulling the elongate wire proximally, the central stop exerts a force on the proximal collar, wherein the force has a directional component in a proximal direction parallel to a longitudinal axis of the elongate wire.

24. The method of claim 17, wherein the medical device further includes an elongate tubular member disposed over the elongate wire proximal of the expandable cage, wherein pulling the elongate wire proximally causes the proximal collar to abut a distal end of the elongate tubular member.
